(19) United States
(12) Patent Application Publication
(10) Pub. No.: US 2011/0008859 A1
(43) Pub. Date: Jan. 13, 2011

(54) Production of Ethanol from Tree Sap

(76) Inventor: Andrew Polczynski, North Huntingdon, PA (US)

Correspondence Address: Andrew Polczynski, 2210 Turkey Farm Road, North Huntingdon, PA, 15642, US

(21) Appl. No.: 12/501,430

(22) Filed: Jul. 11, 2009

(51) Int. Cl. C12P 7/06

(52) U.S. Cl. 435/161

(57) ABSTRACT

The present invention involves an improvement in the production of ethanol by using tree sap as the source of the feedstock for the ethanol production process.

### CROSS REFERENCE TO RELATED APPLICATIONS

Not applicable.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

Not applicable.

### BACKGROUND OF THE INVENTION

1. Field of the Invention

The invention relates generally to a process for producing ethanol, and more specifically, to producing ethanol using the sap of trees as the feedstock for the process.

2. Background of the Invention

Ethanol is a high-octane fuel used primarily as a gasoline additive and extender. Over the past few years, however, surging prices for petroleum-based fuels are dramatically increasing the demand for ethanol and the interest in ethanol production in the United States.

Ethanol is produced primarily from carbohydrates such as sugar and starch by fermentation using yeast or other organisms. Thus far, the production of ethanol known in the art with regard to using these methods is limited to using two primary feedstocks: grain crops (primarily corn and wheat) and sugar crops (sugarcane, sugar beets, or molasses).

Approximately sixty percent of world ethanol production uses sugar crops to as the primary feedstock, with the remaining forty percent using grain crops as the primary feedstock. The choice of feedstock used to produce ethanol is based primarily on the least-expensive feedstock crop available to ethanol producers in a particular country. Currently corn is the dominant feedstock being used in the United States. Corn-based ethanol accounts for more than ninety percent of the total ethanol produced in the United States. Producing ethanol from sugar crops in the United States is not deemed feasible because their use is much more profitable for producing refined sugar.

The growing demand for ethanol as a motor fuel and the primary use of corn for food production are at direct odds with one another. This greater demand has caused an increase in the price of corn which has in turn caused an increase in food prices across the board. A need now exists for a novel source of feedstock for the production of ethanol to meet the increased demand without placing a further strain on food production.

The production of ethanol using tree sap is a more efficient method than the current methods of using corn and sugar cane. The tree sap is collected as a liquid; therefore, the sap does not require the processing of solids as required when using corn, sugar beets, sugar cane, and other biomass. As a result, unlike in the production of ethanol from corn and other biomass, sap does not need to be liquefied to be transformed into a fermentable substance. Also, grain crops such as corn require the addition of enzymes to break down the complex carbohydrates into simpler forms that can be consumed by the yeast. The use of tree sap does not require the addition of any enzymes.

The production of ethanol using tree sap is not an obvious extension of the current art. Entities skilled in the art of producing ethanol from crops would not see tree sap as an obvious source of feedstock because tree sap can be collected from trees that currently have no commercial value. Tree sap is not produced through the planting of a crop that is harvested at the end of a growing season as is sugar cane, sugar beets, corn, and other biomass. Rather, sap is collected from a living tree and may be collected from the same individual tree year after year. The collection and processing of this feedstock is very different from that used by individuals skilled in the current art; and therefore, the production of ethanol using tree sap is not obvious.

### BRIEF SUMMARY OF THE INVENTION

The object of this invention is to produce ethanol using tree sap as the feedstock in conjunction with the use of the unrelated prior art processes of sap collection and ethanol production into one comprehensive ethanol production process. An advantage of the use of tree sap as the feedstock for the production of ethanol is that it will not produce a greater demand on corn, sugar cane, or other crops that have a value other than ethanol production. Additionally, an advantage of this invention over the other inventions related to the uses of tree sap is that any tree species that produces sap may be used because the flavor of the final product is irrelevant. An advantage to this invention is that many species of trees that are not otherwise of commercial value may now have a commercial value. A feature of this process is that unlike feedstock used in the prior art ethanol production processes that use sugar cane and corn, tree sap is a perennial feedstock. That is, it does not need to be sown every season as corn does. Another feature of this process is that grain crops like corn must be processed prior to beginning fermentation. Once harvested, the corn must be milled, cooked, cooled, and then mixed with an enzyme to break down the starches into fermentable sugars. The present invention requires no processing of solid materials as is necessary with all current known crops, requires no additives as is required with grain crops, and does not require annual planting and harvesting as is necessary with both grain and sugar crops. Unlike any prior art, tree sap may be fermented without any processing occurring prior to the addition of yeast.

The present invention relates to a process for producing ethanol using the sap of trees as the source of the feedstock comprising the steps of:

a. Collecting tree sap into a central container.

b. Producing ethanol from the tree sap collected through fermentation and alcohol recovery.

### BRIEF DESCRIPTION OF THE DRAWING

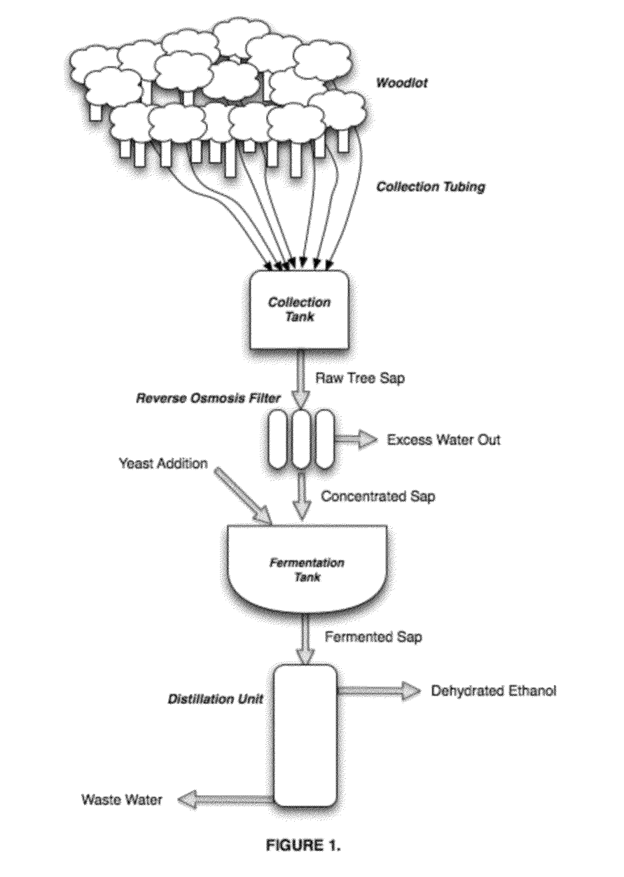

The invention together with the above and other objects and advantages will be best understood from the following detailed description of the preferred embodiment of the invention shown in the accompanying drawing. FIG. 1 depicts an example of a process flow diagram of the system.

### DETAILED DESCRIPTION OF THE INVENTION

The inventor has recognized that several previously unrelated arts may be combined into one for the production of ethanol using the sap of trees as the feedstock.

First, sap must be collected from trees. The most common method for doing so is commonly called “tapping”. “Tap holes” are drilled into a tree. The tap hole allows sap to run from the tree for collection. Typically, the tap holes are fitted with a spout made of metal, plastic, or wood. The spout is in turn connected to a network of tubing that leads to a tank or other container to gather the sap in a central location. Alternately, containers are placed beneath the spouts to collect the sap. The containers are then emptied manually into a central tank or container. Once collected, by any method, the sap must be immediately processed or refrigerated to prevent spoiling.

The next step after collection is to concentrate the sugar content of the sap to a high enough level to begin the fermentation process. The previously known methods for concentration of the sap used during the maple syrup production process are evaporation or a combination of reverse osmosis (RO) filtration followed by evaporation. The purpose of the concentration step is to bring the sugar content of the sap into the range of ten to twenty percent. This is a typical sugar content used when processing feedstock into ethanol.

Once the tree sap has been processed to an adequate sugar concentration, the fermentation step begins. Multiple prior arts have been developed to produce ethanol from sugar-based feedstock. The basis of these processes is almost always fermentation. In fermentation, yeast or similar biota is added to the sugar solution. The yeast organisms consume the sugar. In so doing, they create byproducts consisting primarily of carbon dioxide and ethanol.

The final step of the process is to utilize a prior art of dehydration to recover the ethanol from the remaining water. The fermentation process yields a solution with an ethanol content of at least ten percent. To be suitable for use as a fuel source, the fermented sap must be dehydrated to nearly one hundred percent ethanol. The dehydrated ethanol is the final product of the present invention.

### EXAMPLE

A tap hole is drilled into a tree of any species that is assumed to produce sap. The tap hole is 5/16 of an inch in diameter and approximately two inches deep. A plastic spout is snugly inserted into the tap hole to prevent sap from leaking around the spout. One end of a length of flexible polymer tubing is securely attached to the spout. Other trees are tapped and connected to tubing in a similar manner. The ends of these lengths of tubing not connected to spouts terminate inside a common collection container sized large enough to collect all the sap estimated to flow from the tapped trees. The container is placed lower in elevation than all of the spouts to allow the sap to travel by gravity into the collection container. The tubing from the spouts is installed in a straight line to prevent sags that would result in the entrapment of sap in the sag. When the atmospheric conditions are appropriate, tree sap begins to run. Some of the sap from each tree runs through the collection tubing and into the collection container. Once sap begins to be collected in the container it is pumped through a reverse osmosis (RO) filter and concentrated to a sugar content of fifteen percent. The concentrated sap discharged from the RO filter is directed into a fermentation vat consisting of a 55-gallon plastic drum. The drum is refrigerated to forty degrees Fahrenheit until full of concentrated sap. When this drum is full, the discharge of the RO filter is directed to an empty drum until it is filled, and so on. Once full of sap, yeast is added to each drum to begin the fermentation process, the drum is sealed to prevent air from entering, and a water-trap vent is installed on the top of the drum to permit expanding gases to escape the drum.

The fermentation process continues for several days to completion. The resultant ethanol/water solution is pumped from a fermentation vat into a distillation system. In the distillation process, the solution is heated to 180 degrees Fahrenheit. This temperature is above the boiling point of ethanol, but below the boiling point of water. The ethanol in solution is boiled out of the water into its gaseous form. The gaseous ethanol is condensed in a separate stage of the distillation system. The condensed (liquid) ethanol is then collected in a final container at 99.5 percent ethanol, 0.5 percent water.

Having thereby described the subject matter of the present invention it should be apparent that many substitutions, modifications and variations of the invention are possible in light of the above teachings. It is therefore to be understood that the invention as taught and described herein is only to be limited to the extent of the breadth, and scope of the appended claim.

I claim:

1. A process for fermenting sap from sap producing trees to produce ethanol comprising:
a. collecting the sap from the sap producing trees;
b. processing the sap to produce a concentrated sugar solution;
c. fermenting the concentrated sugar solution; and
d. dehydrating the fermented concentrated sugar solution to recover the ethanol.
